(19) United States
(12) Patent Application Publication
(10) Pub. No.: US 2022/0401589 A1
(43) Pub. Date: Dec. 22, 2022

(54) PRE-TARGETING IMAGING AGENTS

(71) Applicant: Eli Lilly and Company, Indianapolis, IN (US)

(72) Inventors: Aldo CAGNOLINI, West Chester, PA (US); Hui XIONG, Chesterbrook, PA (US)

(21) Appl. No.: 17/777,182

(22) PCT Filed: Dec. 4, 2020
(86) PCT No.: PCT/US2020/063357

§ 371 Date: May 16, 2022

Related U.S. Application Data
(60) Provisional application No. 62/946,218, filed on Dec. 10, 2019.

(51) Int. Cl. A61K 51/04; C07D 257/08

(52) U.S. Cl. CPC A61K 51/044; A61K 51/0495; A61K 51/0497; C07D 257/08

(57) ABSTRACT

The present invention provides a novel compound of the formula: methods of making this compound, methods of using this compound for pretargeted imaging, and preparations of such formulations.

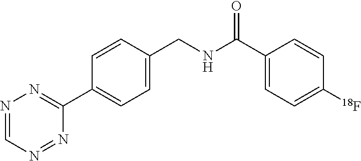 [SMILES: O=C(NCc1ccc(-c2nncnn2)cc1)c1ccc([18F])cc1]

The present invention relates to a compound N-(4-(1,2,4,5-tetrazin-3-yl)benzyl)-4-fluorobenzamide, and the ¹⁸F labelled version of this compound, and to pharmaceutically acceptable salts of these compounds, and to intermediates for preparation of these compounds, and to methods of using these compounds for pretargeted imaging, and to compositions and formulations of these compounds for diagnostic imaging (such as pretargeted imaging), and to methods of pretargeted imaging using these compounds, compositions, and formulations.

Historically, PET imaging using large molecules has been achieved through direct labelling of full-length antibodies. Antibodies possess exquisite specificity and selectivity but are often hindered by their slow clearance. Imaging with antibodies utilizes long-lived radionuclides where imaging is performed after a 7-10 days post injection of the radioimmunoconjugate in order for non-specific background signal to clear. This timeline is not easily incorporated into clinical practice and exposes the patient to unnecessary levels of radioactivity. In order to minimize disruption to normal clinical practice and radioactive exposure to the patient, pretargeted based imaging systems have been developed. This pretargeted approach is a two-step process based on the biorthogonal inverse-electron-demand Diels-Alder (IEDDA) reaction between tetrazines and trans-cyclooctene (TCO) derivatives which takes advantage of the specificity and selectivity of a large molecule and the rapid pharmacokinetics of small molecules with short-lived radionuclides. There are numerous preclinical examples of pretargeted imaging that have been described in the literature for peripheral targets (see J. Med. Chem. 2017, 60, 8201-8217 and J. Label Compd. Radiopharm 2014, 57 285-290).

For antibody-based CNS imaging agents, the blood-brain barrier (BBB) presents an additional challenge. In 2017, Prof. Syvanen and coworkers (Uppsala University) demonstrated improved brain uptake of bispecific antibodies targeting Aβ protofibril, through transferrin receptor (TfR) mediated transport across the blood brain barrier. Subsequent PET imaging studies using ¹²⁴I-labeled antibodies showed differentiated distribution between transgenic and wild-type mice, 3 days post injection. The distribution pattern in various brain regions were in good correlation with Aβ pathology see, Stina Syvänen et al. Theranostics, 2017; 7(2): 308-318.

Beyond brain penetrant large molecules, the other requisite for any successful CNS pretargeted imaging studies is the availability of brain-penetrant, fast-clearing, reactive yet stable, small molecule chasers containing tetrazine reactive groups. Several ¹¹C- and ¹⁸F-labeled small molecule tetrazine chasers have been reported to have appreciable brain uptake (see, Hannes Mikula et al. Bioconjugate Chem. 2016, 27, 7, 1707-1712; Hannes Mikula et al. Angew. Chem. Int. Ed. 2014, 53, 9655-9659). However, their application in CNS pretargeted imaging studies have not been reported.

In 2019, Brendon Cook and collaborators reported the first case of CNS pretargeted imaging to study the distribution of an antisense oligonucleotide (ASO) in rat brain (World Molecular Imaging Congress Conference Poster 139). Rats were intrathecally administered 2.5 mM ASO-TCO conjugate in 30 μL saline solution and followed with an intravenous injection of a CNS-penetrant tetrazine, [¹⁸F]-537-Tz, 24 and 168 hours post ASO-TCO. Static PET-CT scans were performed 75-90 minutes after administration of [¹⁸F]-537-Tz. Higher radiotracer uptake was observed in the brain and spine of the animals that received ASO-TCO relative to the control group. It was also reported that [¹⁸F]-537-Tz showed brain uptake (1.7±0.9% ID by 10 min P.I.) in wild type mice by dynamic PET imaging.

By comparison, dynamic PET imaging of [¹⁸F]—N-(4-(1,2,4,5-tetrazin-3-yl)benzyl)-4-fluorobenzamide in WT mice showed that this compound readily crosses the blood-brain-barrier and reaches a peak brain uptake of 4.1±0.3% ID/g approximately 2.5 min post injection, followed by a steady clearance of tracer to 0.8±0.1% ID/g by 60 minutes. An agent that possesses robust brain penetration and rapid and complete washout from the brain provides a larger window to achieve higher signal to background ratios resulting in better image quality. Thus, we expect [¹⁸F]—N-(4-(1,2,4,5-tetrazin-3-yl)benzyl)-4-fluorobenzamide to provide an advantage for pretargeted CNS imaging.

The present embodiments provide novel compounds, compositions, formulations and methods for pretargeted imaging. This type of improved technology advancing the capacity to image patients is thus also needed to expand the clinical benefits and impact of diagnostic imaging. An improved imaging agent will provide enhanced pretargeted images, as compared with currently known agents.

The present embodiments also provide the compound N-(4-(1,2,4,5-tetrazin-3-yl)benzyl)-4-fluorobenzamide, also referred to herein as “Compound 1”, which can be structurally represented as the compound of Formula I:

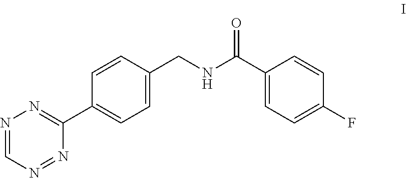 [SMILES: O=C(NCc1ccc(-c2nncnn2)cc1)c1ccc(F)cc1]

The compound of Formula I may be a free base, as shown above, or may be a pharmaceutically acceptable salt.

The present embodiments provide the compound [¹⁸F]—N-(4-(1,2,4,5-tetrazin-3-yl)benzyl)-4-fluorobenzamide, also referred to herein as “Compound 2”, which can be structurally represented as the compound of Formula II:

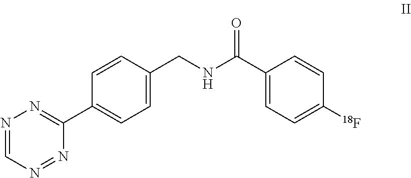 [SMILES: O=C(NCc1ccc(-c2nncnn2)cc1)c1ccc([18F])cc1]

The compound of Formula II may be a free base, as shown above, or may be a pharmaceutically acceptable salt.

The present embodiments provide for pharmaceutically acceptable salts of either Compound 1 or Compound 2 or the use of the compounds as the free base.

The present embodiments further provide the use of the compound of Formula I and/or the compound of Formula II, and/or mixtures thereof, for the preparation of imaging agents, such as, for example, pretargeted imaging agents.

The present embodiments provide for the use of compounds of Formula I or II, for the manufacture of a radiopharmaceutical agent for imaging (pretargeted imaging) in humans. In another aspect the invention provides methods of preparing compounds of Formula I or II.

In another aspect the present embodiments provide a pharmaceutical composition comprising Compound 1 or Compound 2, or pharmaceutically acceptable salt thereof, which is formulated in ethanol (such as, for example 10% EtOH (v/v)) and buffer (which may be PBS buffer), preferably for use in humans. It should also be noted that, in some embodiments, the formulation does not include ascorbate or ascorbic acid, as it has been found that the tetrazine moiety in Formula I and Formula II could be readily reduced by ascorbate formulation. Thus, although ascorbate formulation is readily used in many formulations for imaging, it may not be suitable for imaging with the compounds of Formula I or Formula II.

The present invention also provides methods for pretargeted imaging comprising introducing into a patient a detectable quantity of Compound 1 or 2, or pharmaceutically acceptable salt thereof, or a composition thereof.

The following Schemes, Preparations, and Examples are provided to better elucidate the practice of the present invention. Suitable reaction conditions for the steps of these Schemes, Preparations, and Examples are well known in the art and appropriate modification of reaction conditions, including substitution of solvents and co-reagents are within the ability of the skilled artisan.

Furthermore, the skilled artisan will appreciate that in some circumstances, the order in which moieties are introduced is not critical. The particular order of steps required to produce the compounds of Formula I or Formula II is dependent upon the particular compound being synthesized, the starting compound, and the relative lability of the substituted moieties, as is well appreciated by the skilled chemist. The skilled artisan will appreciate that not all substituents are compatible with all reaction conditions. These compounds may be protected or modified at a convenient point in the synthesis by methods well known in the art. The intermediates and final products of the present invention may be further purified, if desired by common techniques such as recrystallization or chromatography over solid supports such as silica gel or alumina.

The compounds of the present invention are preferably formulated as radiopharmaceutical compositions administered by a variety of routes. Preferably, such compositions are for intravenous use, preferably in humans. Such pharmaceutical compositions and processes for preparing same are well known in the art. See, e.g., Remington: The Science and Practice of Pharmacy (P. P. Gerbino, 21^{st} ed., Lippincott Williams & Wilkins, 2006).

Preferred formulations may be preparations of Compound 1 or Compound 2. Particularly preferred is Compound 1 or Compound 2 prepared according to the procedures described herein. A preferred formulation of Compound 1 or Compound 2 is formulated in ethanol, such as for example, 10% EtOH (v/v). This formulation may also include a buffer, such as a PBS buffer. Other ingredients may also be used.

Compounds of Formula I and II have been discovered to be surprisingly and unexpectedly advantageous for pretargeted imaging, preferably including human clinical imaging. In some embodiments, the Compounds of Formula I and II may be used for pre-targeted imaging. For example, the compounds of Formula I and II may be brain penetrant, and thus may be used as a tracer for CNS (central nervous system) pre-targeted imaging. In some embodiments, the pretargeted imaging of CNS targets may be achieved in 3 or 4 steps:

1. I.V. (intravenous) or I.T (intrathecal) administration of biologics-TCO conjugate (for example, shuttled bispecific antibody-TCO conjugate, or oligonucleotide-TCO conjugate);

2. Wait for sufficient time (days) to allow distribution and systematic clearance of biologics-TCO conjugate

3. An optional step of I.V. injection of periphery-restricted tetrazines to mask peripherally circulating biologics-TCO conjugate;

4. I.V. administration of a brain penetrant compound of Formula I or Formula II, followed by brain PET imaging.

With respect to the preparation of biologics-TCO conjugate, the use of such technology is published in the literature (see below) and is known for oncology pretargeted imaging studies):
1. Bioconjugate Chem. 2018, 29, 538-545 (TCO antibody conjugation; Use of clearing agent to mask circulating antibody-TCO)
2. Bioconjugate Chem. 2013, 24, 1210-1217 (TCO antibody conjugation)
J. Med. Chem. 2017, 60, 8201-8217 (TCO antibody conjugation, Pretargeting Radioligand Optimization)

For oligonucleotide-TCO conjugation, this type of conjugate (a TCO-PEG4 Oligo Modification) is commercially available from the Bio-Synthesis company of Lewisville, Tex., USA. Thus, those skilled in the art will appreciate how to accomplish the preparation of the biologics-TCO conjugate.

Some of the potential benefits of pretargeted imaging include: the ability to use short-lived radionuclides by separating the delivery of the radioactivity from the targeting vector such as biologics with high specificity and selectivity and the ability to reduce patient exposure to radioactivity.

Certain abbreviations may be used below. These abbreviations mean as follows: “CAS #” refers to Chemical Abstracts Registry number; “Ci” refers to Curie or Curies; “CT” refers to computed tomography; “δ” refers to chemical shift in nuclear magnetic resonance spectroscopy; “DMF” refers to N,N-dimethylformamide; “DMSO” refers to dimethyl sulfoxide; “ES/MS” refers to electrospray-mass spectrometry; “HPLC” refers to High Performance Liquid Chromatography; “min” refers to minute or minutes; “mCi” refers to milliCurie or milliCuries; “MHz” refers to megahertz; “μL” refers to microliter or microliters; “N” refers to number of replications or sample size in an experiment; “NMR” refers to nuclear magnetic resonance; “PET” refers to positron emission tomography” “OAc” refers to acetate; “ppm” refers to parts per million; “SEM” refers to standard error of the mean; “t_{R}” refers to retention time; “WT” refers to wild type.

### General Chemistry and Preparations

The following Preparations and Examples further illustrate the invention and represent typical synthesis of the compounds of the invention. The reagents and starting materials are readily available or may be readily synthesized by one of ordinary skill in the art. It should be understood that the Examples are set forth by way of illustration and not limitation, and that various modifications may be made by one of ordinary skill in the art.

NMR spectroscopy is performed on a Bruker AVIII HD 400 MHz NMR Spectrometer, obtained as a DMSO-d₆ solution reported in ppm, using residual solvent [DMSO-d₆, 2.50 ppm] as reference standard. When peak multiplicities are reported, the following abbreviations may be used: s (singlet), d (doublet), t (triplet), m (multiplet), Coupling constants (J), when reported, are reported in hertz (Hz).

ES/MS is performed on a Waters® Acquity UPLC system. Electrospray mass spectrometry measurements (acquired in positive and/or negative mode) are performed on a Waters® Acquity QDa mass detector interfaced to the UPLC system. LC-MS conditions: column: Waters Acquity UPLC® BEH 2.1×30 mm, 1.7μ; gradient: 10-98% B in 3 min, hold 98% B for 0.5 min, and then return to 10% B for 0.6 min; column temperature: 40° C.+/−10° C.; flow rate: 1.2 mL/min; Solvent A: deionized water with 0.1% HCOOH; Solvent B: 100% acetonitrile; wavelength 250-650 nm.

HRMS data were obtained on a Waters QTof mass spectrometer using an electrospray ionization positive scan mode. Nominal resolution MS data were obtained on a Waters Micromass ZQ mass spectrometer using an ESI positive ionization scan mode.

Of course, other instruments and means of detecting and for ES/MS are known in the art and would be known by a person of ordinary skill.

Preparative and analytical HPLC conditions, when used, are detailed below.

### EXAMPLE 1

### N-(4-(1,2,4,5-tetrazin-3-yl)benzyl)-4-fluorobenzamide

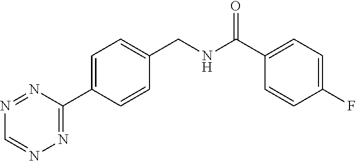 [SMILES: O=C(NCc1ccc(-c2nncnn2)cc1)c1ccc(F)cc1]

To a stirred suspension of [4-(1,2,4,5-tetrazin-3-yl)phenyl]methanamine hydrochloride (90 mg, 0.4 mmol, commercially available from Click Chemistry Tools, CAS #1416711-59-5), Polymer Supported triethylamine (400 mg, 1.2 mmol) in dichloromethane (10 mL) is added dropwise a solution of 4-fluorobenzoyl chloride (70 mg, 0.44 mmol, commercially available from Sigma-Aldrich, CAS #403-43-0) in dichloromethane (2 mL). The reaction mixture is stirred at room temperature for 30 min, filtered, and concentrated under reduced pressure. The resulting residue is purified by chromatography on silica gel, eluting with a gradient of 0-60% dichloromethane/ethyl acetate, to afford the title compound as a purple solid (60 mg, 48% yield) after evaporation of the desired chromatographic fractions. ¹H NMR (400.13 MHz, DMSO-d₆) δ ppm: 4.62 (d, J=6.0 Hz, 2H), 7.35-7.30 (m, 2H), 7.62 (d, J=8.7 Hz, 2H), 8.02-7.98 (m, 2H), 8.49-8.46 (m, 2H), 9.16 (t, J=6.0 Hz, 1H), 10.57 (s, 1H). ¹⁹F NMR (376.45 MHz, DMSO-d₆) δ ppm: −109.3. ES/MS m/z 310 (M+H).

### EXAMPLE 2

[¹⁸F]—N-(4-(1,2,4,5-tetrazin-3-yl)benzyl)-4-fluorobenzamide

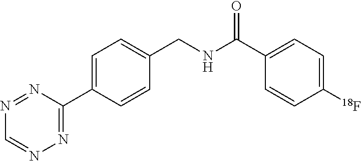 [SMILES: O=C(NCc1ccc(-c2nncnn2)cc1)c1ccc([18F])cc1]

A typical radiochemical yield of the title compound is 3% (using 0.7-3.7 Ci starting activity) with a total synthesis time of 120-130 min. The intermediate N-succinimidyl 4-[¹⁸F]fluorobenzoate is prepared according to literature methods (see Wüst, F., Hultsch, C., Bergmann, R., Johannsen, B., Henle, T., 2003; Radiolabelling of isopeptide N-ε-(γ-glutamyl)-L-lysine by conjugation with N-succinimidyl-4-[18F]fluorobenzoate. Appl. Radiat. Isot. 59, 43-48) adapted onto a TRACERlab FX_{F-N}. [¹⁸F]fluorobenzoate (0.15-0.51 Ci) in acetonitrile (1 mL) is dried under a stream of nitrogen at 60° C. and to the resulting residue, a solution of [4-(1,2,4,5-tetrazin-3-yl)phenyl]methanamine hydrochloride (5 mg, 26.5 μmol) and triethylamine (29.1 mg, 287 μmol) in anhydrous DMF (1 mL) are added and the resulting solution is kept at room temperature for 15 min with occasional shaking. The crude reaction is diluted with water (2 mL) and purified by semi-preparative HPLC (Agilent ZORBAX Eclipse XDB-C18, 4×250 mm, 5 μm, 55% 20 mM NH₄OAc in water/45% acetonitrile, Flow rate 4 mL/min, 280 nm). The fraction containing the purified title compound is diluted with water (40 mL) and reconstituted into a formulation of 10% (v/v) EtOH in 0.9% saline (10 mL) for subsequent use. The amount of specific radioactivity of the obtained title compound ranges from 0.015 to 0.044 Ci.

PET-CT imaging of [¹⁸F]—N-(4-(1,2,4,5-tetrazin-3-yl)benzyl)-4-fluorobenzamide

A Siemens Inveon® Multimodality Scanner (Siemens, Germany) is used for micro PET/CT imaging. Male CD-1 (6-week-old, ˜30 g) mice are anesthetized with 3% isoflurane/97% oxygen and placed on the bed of the scanner. The mice are administered [¹⁸F]—N-(4-(1,2,4,5-tetrazin-3-yl)benzyl)-4-fluorobenzamide via a bolus intravenous tail vein injection (˜300 Ci in a total volume of 200 μL saline). A total of six (four 60 min and two 90 min) dynamic PET scans are conducted, followed by a short high-resolution CT scan for anatomical registration. PET images are generated for each minute of the acquisition time. Uptake of the tracer in the brain, muscle, and bone are determined by visually drawing regions of interest (ROIs) based on the fused PET/CT images and the corresponding activity values are determined using the Inveon® Research Workplace software. All values are represented as % injected dose per gram (% ID/g).

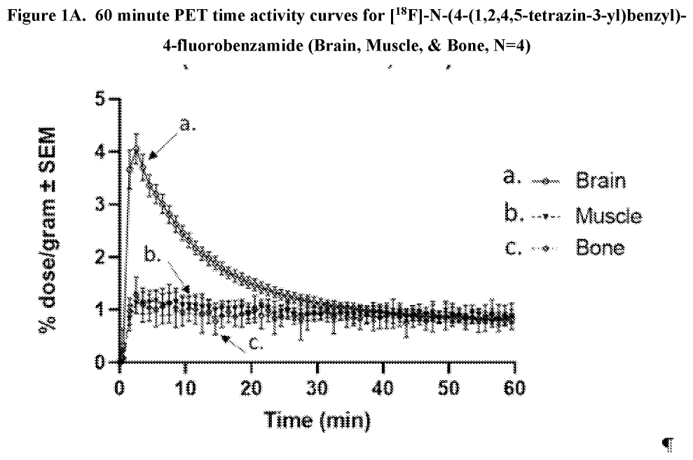

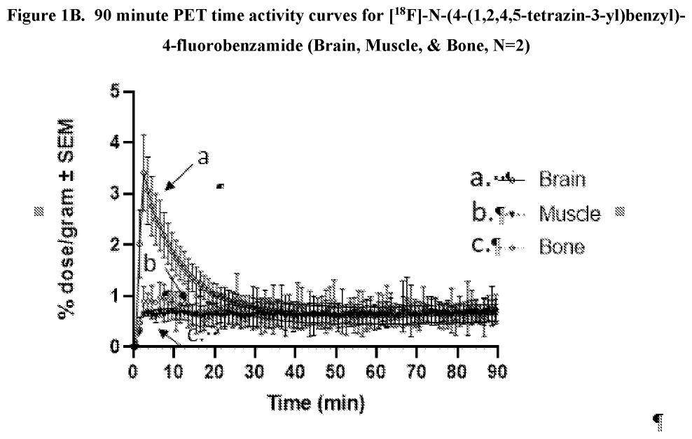

Analysis of the four 60-minute PET scans indicate that [¹⁸F]—N-(4-(1,2,4,5-tetrazin-3-yl)benzyl)-4-fluorobenzamide readily crosses the blood-brain-barrier. Peak brain uptake of 4.1% ID/g is observed at 2.5 min post injection followed by a steady clearance of tracer to 0.8% ID/g at 59.5 min. Uptake of [¹′F]—N-(4-(1,2,4,5-tetrazin-3-yl)benzyl)-4-fluorobenzamide is also observed throughout the body in organs such as the liver, kidneys and heart. Bone uptake throughout the scan period remained low, consistent with an absence of defluorination for this tracer. Results of the 90 min scans are similar to the 60 min scan results with peak brain uptake observed at 3.4% ID/g at 2.5 min post injection followed by an immediate wash out to 0.6% ID/g by 59.5 min. Residual uptake is also observed throughout the body of the mouse. Time activity curves are generated for [¹⁸F]—N-(4-(1,2,4,5-tetrazin-3-yl)benzyl)-4-fluorobenzamide in CD-1 male mice (n=4). Data is shown in graphical format in FIGS. 1A and 1B and in tabular format below.

FIG. 1A is a graph of 60 minute PET time activity curves for [¹⁸F]—N-(4-(1,2,4,5-tetrazin-3-yl)benzyl)-4-fluorobenzamide (Brain, Muscle, & Bone, N=4)

FIG. 1B is a graph of 90 minute PET time activity curves for [¹⁸F]—N-(4-(1,2,4,5-tetrazin-3-yl)benzyl)-4-fluorobenzamide (Brain, Muscle, & Bone, N=2)

TABLE 1A
60 minute PET time activity table for [¹⁸F]-N-(4-(1,2,4,5-tetrazin-3-
yl)benzyl)-4-fluorobenzamide (Brain, Muscle, & Bone)

| Time | Brain | | | Muscle | | | Bone | | |
|---|---|---|---|---|---|---|---|---|---|
| (min) | Mean | SEM | N | Mean | SEM | N | Mean | SEM | N |
| 0 | 0 | 0 | 4 | 0 | 0 | 4 | 0 | 0 | 4 |
| 0.5 | 0.32 | 0.32 | 4 | 0.02 | 0.02 | 4 | 0.08 | 0.08 | 4 |
| 1.5 | 3.67 | 0.37 | 4 | 0.84 | 0.24 | 4 | 0.97 | 0.12 | 4 |
| 2.5 | 4.06 | 0.28 | 4 | 1.14 | 0.20 | 4 | 1.28 | 0.17 | 4 |
| 3.5 | 3.70 | 0.25 | 4 | 1.13 | 0.21 | 4 | 1.09 | 0.16 | 4 |
| 4.5 | 3.36 | 0.21 | 4 | 1.15 | 0.22 | 4 | 1.04 | 0.13 | 4 |
| 5.5 | 3.20 | 0.18 | 4 | 1.17 | 0.21 | 4 | 1.01 | 0.17 | 4 |
| 6.5 | 3.01 | 0.18 | 4 | 1.10 | 0.21 | 4 | 1.07 | 0.17 | 4 |
| 7.5 | 2.81 | 0.17 | 4 | 1.11 | 0.20 | 4 | 1.12 | 0.18 | 4 |
| 8.5 | 2.62 | 0.15 | 4 | 1.15 | 0.19 | 4 | 1.04 | 0.19 | 4 |
| 9.5 | 2.46 | 0.14 | 4 | 1.09 | 0.19 | 4 | 0.97 | 0.14 | 4 |
| 10.5 | 2.32 | 0.14 | 4 | 1.09 | 0.16 | 4 | 1.03 | 0.12 | 4 |
| 11.5 | 2.17 | 0.12 | 4 | 1.08 | 0.17 | 4 | 1.01 | 0.14 | 4 |
| 12.5 | 2.07 | 0.13 | 4 | 1.06 | 0.17 | 4 | 0.93 | 0.19 | 4 |
| 13.5 | 1.97 | 0.13 | 4 | 1.06 | 0.15 | 4 | 0.96 | 0.11 | 4 |
| 14.5 | 1.88 | 0.12 | 4 | 1.00 | 0.13 | 4 | 0.78 | 0.13 | 4 |
| 15.5 | 1.79 | 0.12 | 4 | 1.02 | 0.14 | 4 | 0.89 | 0.11 | 4 |
| 16.5 | 1.70 | 0.11 | 4 | 1.02 | 0.15 | 4 | 0.96 | 0.10 | 4 |
| 17.5 | 1.64 | 0.11 | 4 | 1.03 | 0.15 | 4 | 0.87 | 0.09 | 4 |
| 18.5 | 1.58 | 0.11 | 4 | 1.05 | 0.14 | 4 | 0.92 | 0.12 | 4 |
| 19.5 | 1.52 | 0.11 | 4 | 0.94 | 0.12 | 4 | 0.93 | 0.10 | 4 |
| 20.5 | 1.47 | 0.11 | 4 | 0.95 | 0.11 | 4 | 1.01 | 0.13 | 4 |
| 21.5 | 1.42 | 0.10 | 4 | 1.06 | 0.14 | 4 | 0.88 | 0.12 | 4 |
| 22.5 | 1.38 | 0.11 | 4 | 1.04 | 0.15 | 4 | 0.99 | 0.11 | 4 |
| 23.5 | 1.33 | 0.10 | 4 | 0.92 | 0.13 | 4 | 0.85 | 0.16 | 4 |
| 24.5 | 1.29 | 0.10 | 4 | 0.96 | 0.13 | 4 | 0.88 | 0.10 | 4 |
| 25.5 | 1.26 | 0.10 | 4 | 0.97 | 0.15 | 4 | 1.02 | 0.08 | 4 |
| 26.5 | 1.23 | 0.10 | 4 | 0.92 | 0.12 | 4 | 0.88 | 0.08 | 4 |
| 27.5 | 1.18 | 0.10 | 4 | 0.89 | 0.13 | 4 | 0.83 | 0.20 | 4 |
| 28.5 | 1.17 | 0.10 | 4 | 0.91 | 0.12 | 4 | 0.92 | 0.15 | 4 |
| 29.5 | 1.14 | 0.10 | 4 | 0.94 | 0.12 | 4 | 0.93 | 0.07 | 4 |
| 30.5 | 1.11 | 0.10 | 4 | 0.93 | 0.12 | 4 | 0.92 | 0.05 | 4 |
| 31.5 | 1.09 | 0.09 | 4 | 0.99 | 0.14 | 4 | 0.96 | 0.09 | 4 |
| 32.5 | 1.07 | 0.10 | 4 | 0.89 | 0.12 | 4 | 0.85 | 0.16 | 4 |
| 33.5 | 1.04 | 0.09 | 4 | 0.93 | 0.12 | 4 | 0.95 | 0.07 | 4 |
| 34.5 | 1.04 | 0.10 | 4 | 0.92 | 0.11 | 4 | 0.87 | 0.10 | 4 |
| 35.5 | 1.03 | 0.10 | 4 | 0.91 | 0.16 | 4 | 0.84 | 0.07 | 4 |
| 36.5 | 1.00 | 0.10 | 4 | 0.93 | 0.14 | 4 | 0.84 | 0.11 | 4 |
| 37.5 | 0.98 | 0.09 | 4 | 0.91 | 0.15 | 4 | 0.90 | 0.09 | 4 |
| 38.5 | 0.97 | 0.09 | 4 | 0.93 | 0.15 | 4 | 0.87 | 0.13 | 4 |
| 39.5 | 0.97 | 0.10 | 4 | 0.97 | 0.14 | 4 | 0.85 | 0.11 | 4 |
| 40.5 | 0.95 | 0.09 | 4 | 0.93 | 0.14 | 4 | 0.89 | 0.12 | 4 |
| 41.5 | 0.93 | 0.09 | 4 | 0.89 | 0.14 | 4 | 0.93 | 0.14 | 4 |
| 42.5 | 0.93 | 0.09 | 4 | 0.95 | 0.17 | 4 | 0.88 | 0.10 | 4 |
| 43.5 | 0.91 | 0.09 | 4 | 0.88 | 0.11 | 4 | 0.75 | 0.14 | 4 |
| 44.5 | 0.90 | 0.09 | 4 | 0.91 | 0.12 | 4 | 0.87 | 0.10 | 4 |
| 45.5 | 0.89 | 0.09 | 4 | 0.95 | 0.16 | 4 | 0.86 | 0.07 | 4 |
| 46.5 | 0.87 | 0.09 | 4 | 0.92 | 0.13 | 4 | 0.79 | 0.09 | 4 |
| 47.5 | 0.87 | 0.09 | 4 | 0.95 | 0.15 | 4 | 0.87 | 0.15 | 4 |
| 48.5 | 0.86 | 0.09 | 4 | 0.86 | 0.12 | 4 | 0.86 | 0.05 | 4 |
| 49.5 | 0.86 | 0.09 | 4 | 0.99 | 0.17 | 4 | 0.87 | 0.12 | 4 |
| 50.5 | 0.85 | 0.09 | 4 | 0.91 | 0.13 | 4 | 0.90 | 0.11 | 4 |
| 51.5 | 0.85 | 0.09 | 4 | 0.88 | 0.12 | 4 | 0.92 | 0.06 | 4 |
| 52.5 | 0.84 | 0.08 | 4 | 0.81 | 0.10 | 4 | 0.85 | 0.12 | 4 |
| 53.5 | 0.85 | 0.09 | 4 | 0.96 | 0.13 | 4 | 0.85 | 0.05 | 4 |
| 54.5 | 0.84 | 0.09 | 4 | 0.91 | 0.09 | 4 | 0.80 | 0.11 | 4 |
| 55.5 | 0.83 | 0.09 | 4 | 0.87 | 0.10 | 4 | 0.85 | 0.13 | 4 |
| 56.5 | 0.82 | 0.09 | 4 | 0.91 | 0.11 | 4 | 0.93 | 0.13 | 4 |
| 57.5 | 0.82 | 0.09 | 4 | 0.82 | 0.14 | 4 | 0.84 | 0.13 | 4 |
| 58.5 | 0.81 | 0.09 | 4 | 0.83 | 0.11 | 4 | 0.89 | 0.10 | 4 |
| 59.5 | 0.82 | 0.09 | 4 | 0.88 | 0.12 | 4 | 0.88 | 0.12 | 4 |

TABLE 1B
90-minute PET time activity table for [¹⁸F]-N-(4-(1,2,4,5-tetrazin-3-
yl)benzyl)-4-fluorobenzamide (Brain, Muscle, & Bone, % ID/g ± SEM)

| Time | Brain | | | Muscle | | | Bone | | |
|---|---|---|---|---|---|---|---|---|---|
| (min) | Mean | SEM | N | Mean | SEM | N | Mean | SEM | N |
| 0 | 0 | 0 | 2 | 0 | 0 | 2 | 0 | 0 | 2 |
| 0.5 | 0 | 0 | 2 | 0 | 0 | 2 | 0 | 0 | 2 |
| 1.5 | 2.02 | 0.66 | 2 | 0.29 | 0.05 | 2 | 0.48 | 0.10 | 2 |
| 2.5 | 3.41 | 0.74 | 2 | 0.63 | 0.02 | 2 | 0.90 | 0.29 | 2 |
| 3.5 | 3.06 | 0.66 | 2 | 0.66 | 0.05 | 2 | 0.90 | 0.15 | 2 |
| 4.5 | 2.76 | 0.58 | 2 | 0.63 | 0.08 | 2 | 0.90 | 0.30 | 2 |
| 5.5 | 2.50 | 0.50 | 2 | 0.60 | 0.13 | 2 | 0.87 | 0.02 | 2 |
| 6.5 | 2.38 | 0.49 | 2 | 0.68 | 0.09 | 2 | 0.97 | 0.30 | 2 |
| 7.5 | 2.19 | 0.44 | 2 | 0.62 | 0.13 | 2 | 0.94 | 0.29 | 2 |
| 8.5 | 2.02 | 0.40 | 2 | 0.68 | 0.17 | 2 | 0.93 | 0.17 | 2 |
| 9.5 | 1.87 | 0.37 | 2 | 0.71 | 0.17 | 2 | 1.01 | 0.35 | 2 |
| 10.5 | 1.74 | 0.34 | 2 | 0.65 | 0.12 | 2 | 0.70 | 0.38 | 2 |
| 11.5 | 1.63 | 0.33 | 2 | 0.66 | 0.19 | 2 | 0.82 | 0.28 | 2 |
| 12.5 | 1.55 | 0.32 | 2 | 0.67 | 0.14 | 2 | 0.84 | 0.17 | 2 |
| 13.5 | 1.44 | 0.29 | 2 | 0.65 | 0.14 | 2 | 0.83 | 0.45 | 2 |
| 14.5 | 1.36 | 0.27 | 2 | 0.64 | 0.19 | 2 | 0.63 | 0.21 | 2 |
| 15.5 | 1.29 | 0.27 | 2 | 0.64 | 0.21 | 2 | 0.62 | 0.36 | 2 |
| 16.5 | 1.23 | 0.25 | 2 | 0.60 | 0.20 | 2 | 0.71 | 0.42 | 2 |
| 17.5 | 1.16 | 0.24 | 2 | 0.62 | 0.20 | 2 | 0.51 | 0.19 | 2 |
| 18.5 | 1.11 | 0.23 | 2 | 0.63 | 0.22 | 2 | 0.71 | 0.15 | 2 |
| 19.5 | 1.04 | 0.21 | 2 | 0.63 | 0.22 | 2 | 0.74 | 0.34 | 2 |
| 20.5 | 1.02 | 0.22 | 2 | 0.66 | 0.21 | 2 | 0.63 | 0.36 | 2 |
| 21.5 | 0.97 | 0.20 | 2 | 0.61 | 0.13 | 2 | 0.70 | 0.35 | 2 |
| 22.5 | 0.95 | 0.22 | 2 | 0.61 | 0.21 | 2 | 0.69 | 0.34 | 2 |
| 23.5 | 0.91 | 0.20 | 2 | 0.65 | 0.18 | 2 | 0.78 | 0.33 | 2 |
| 24.5 | 0.88 | 0.20 | 2 | 0.64 | 0.25 | 2 | 0.73 | 0.34 | 2 |
| 25.5 | 0.85 | 0.19 | 2 | 0.62 | 0.19 | 2 | 0.95 | 0.49 | 2 |
| 26.5 | 0.83 | 0.18 | 2 | 0.58 | 0.21 | 2 | 0.64 | 0.28 | 2 |
| 27.5 | 0.82 | 0.19 | 2 | 0.60 | 0.21 | 2 | 0.69 | 0.20 | 2 |
| 28.5 | 0.81 | 0.19 | 2 | 0.62 | 0.21 | 2 | 0.60 | 0.21 | 2 |
| 29.5 | 0.78 | 0.18 | 2 | 0.63 | 0.23 | 2 | 0.76 | 0.42 | 2 |
| 30.5 | 0.76 | 0.18 | 2 | 0.64 | 0.24 | 2 | 0.76 | 0.39 | 2 |
| 31.5 | 0.75 | 0.18 | 2 | 0.66 | 0.19 | 2 | 0.60 | 0.35 | 2 |
| 32.5 | 0.74 | 0.20 | 2 | 0.63 | 0.24 | 2 | 0.70 | 0.41 | 2 |
| 33.5 | 0.72 | 0.18 | 2 | 0.62 | 0.25 | 2 | 0.67 | 0.29 | 2 |
| 34.5 | 0.70 | 0.17 | 2 | 0.60 | 0.24 | 2 | 0.74 | 0.48 | 2 |
| 35.5 | 0.70 | 0.17 | 2 | 0.65 | 0.25 | 2 | 0.69 | 0.24 | 2 |
| 36.5 | 0.70 | 0.18 | 2 | 0.65 | 0.25 | 2 | 0.79 | 0.43 | 2 |
| 37.5 | 0.68 | 0.17 | 2 | 0.59 | 0.27 | 2 | 0.62 | 0.40 | 2 |
| 38.5 | 0.68 | 0.17 | 2 | 0.63 | 0.24 | 2 | 0.87 | 0.16 | 2 |
| 39.5 | 0.66 | 0.17 | 2 | 0.61 | 0.23 | 2 | 0.60 | 0.29 | 2 |
| 40.5 | 0.66 | 0.17 | 2 | 0.61 | 0.27 | 2 | 0.82 | 0.44 | 2 |
| 41.5 | 0.66 | 0.17 | 2 | 0.59 | 0.25 | 2 | 0.77 | 0.40 | 2 |
| 42.5 | 0.65 | 0.18 | 2 | 0.63 | 0.20 | 2 | 0.61 | 0.23 | 2 |
| 43.5 | 0.64 | 0.17 | 2 | 0.64 | 0.22 | 2 | 0.65 | 0.44 | 2 |
| 44.5 | 0.64 | 0.17 | 2 | 0.66 | 0.20 | 2 | 0.61 | 0.39 | 2 |
| 45.5 | 0.64 | 0.17 | 2 | 0.63 | 0.20 | 2 | 0.66 | 0.30 | 2 |
| 46.5 | 0.64 | 0.17 | 2 | 0.64 | 0.22 | 2 | 0.67 | 0.32 | 2 |
| 47.5 | 0.64 | 0.17 | 2 | 0.65 | 0.27 | 2 | 0.69 | 0.43 | 2 |
| 48.5 | 0.62 | 0.16 | 2 | 0.61 | 0.25 | 2 | 0.76 | 0.42 | 2 |
| 49.5 | 0.62 | 0.16 | 2 | 0.62 | 0.20 | 2 | 0.91 | 0.40 | 2 |
| 50.5 | 0.61 | 0.16 | 2 | 0.60 | 0.22 | 2 | 0.58 | 0.28 | 2 |
| 51.5 | 0.63 | 0.17 | 2 | 0.59 | 0.20 | 2 | 0.70 | 0.43 | 2 |
| 52.5 | 0.63 | 0.16 | 2 | 0.65 | 0.18 | 2 | 0.51 | 0.27 | 2 |
| 53.5 | 0.62 | 0.17 | 2 | 0.64 | 0.24 | 2 | 0.68 | 0.27 | 2 |
| 54.5 | 0.63 | 0.16 | 2 | 0.69 | 0.27 | 2 | 0.55 | 0.28 | 2 |
| 55.5 | 0.61 | 0.16 | 2 | 0.63 | 0.20 | 2 | 0.68 | 0.42 | 2 |
| 56.5 | 0.62 | 0.17 | 2 | 0.67 | 0.22 | 2 | 0.65 | 0.44 | 2 |
| 57.5 | 0.63 | 0.17 | 2 | 0.63 | 0.29 | 2 | 0.62 | 0.32 | 2 |
| 58.5 | 0.63 | 0.17 | 2 | 0.67 | 0.25 | 2 | 0.67 | 0.31 | 2 |
| 59.5 | 0.62 | 0.16 | 2 | 0.63 | 0.17 | 2 | 0.58 | 0.25 | 2 |
| 60.5 | 0.62 | 0.16 | 2 | 0.63 | 0.24 | 2 | 0.70 | 0.35 | 2 |
| 61.5 | 0.62 | 0.17 | 2 | 0.65 | 0.22 | 2 | 0.76 | 0.26 | 2 |
| 62.5 | 0.62 | 0.16 | 2 | 0.65 | 0.24 | 2 | 0.79 | 0.41 | 2 |
| 63.5 | 0.62 | 0.17 | 2 | 0.69 | 0.28 | 2 | 0.79 | 0.27 | 2 |
| 64.5 | 0.62 | 0.17 | 2 | 0.57 | 0.18 | 2 | 0.65 | 0.20 | 2 |
| 65.5 | 0.62 | 0.17 | 2 | 0.63 | 0.21 | 2 | 0.66 | 0.12 | 2 |
| 66.5 | 0.62 | 0.17 | 2 | 0.69 | 0.30 | 2 | 0.79 | 0.50 | 2 |
| 67.5 | 0.63 | 0.18 | 2 | 0.70 | 0.26 | 2 | 0.89 | 0.47 | 2 |
| 68.5 | 0.64 | 0.17 | 2 | 0.64 | 0.21 | 2 | 0.71 | 0.51 | 2 |
| 69.5 | 0.64 | 0.17 | 2 | 0.63 | 0.24 | 2 | 0.68 | 0.25 | 2 |
| 70.5 | 0.63 | 0.17 | 2 | 0.61 | 0.28 | 2 | 0.53 | 0.13 | 2 |
| 71.5 | 0.63 | 0.17 | 2 | 0.67 | 0.20 | 2 | 0.82 | 0.36 | 2 |
| 72.5 | 0.64 | 0.18 | 2 | 0.65 | 0.18 | 2 | 0.74 | 0.23 | 2 |
| 73.5 | 0.64 | 0.17 | 2 | 0.68 | 0.26 | 2 | 0.73 | 0.26 | 2 |
| 74.5 | 0.65 | 0.17 | 2 | 0.68 | 0.25 | 2 | 0.72 | 0.25 | 2 |
| 75.5 | 0.66 | 0.18 | 2 | 0.63 | 0.25 | 2 | 0.67 | 0.20 | 2 |
| 76.5 | 0.65 | 0.18 | 2 | 0.68 | 0.18 | 2 | 0.58 | 0.24 | 2 |
| 77.5 | 0.65 | 0.18 | 2 | 0.66 | 0.21 | 2 | 0.63 | 0.28 | 2 |
| 78.5 | 0.64 | 0.17 | 2 | 0.63 | 0.16 | 2 | 0.71 | 0.44 | 2 |
| 79.5 | 0.65 | 0.17 | 2 | 0.69 | 0.15 | 2 | 0.51 | 0.27 | 2 |
| 80.5 | 0.65 | 0.17 | 2 | 0.66 | 0.19 | 2 | 0.70 | 0.29 | 2 |
| 81.5 | 0.66 | 0.17 | 2 | 0.66 | 0.21 | 2 | 0.56 | 0.09 | 2 |
| 82.5 | 0.65 | 0.17 | 2 | 0.68 | 0.21 | 2 | 0.76 | 0.53 | 2 |
| 83.5 | 0.65 | 0.18 | 2 | 0.68 | 0.23 | 2 | 0.77 | 0.33 | 2 |
| 84.5 | 0.66 | 0.17 | 2 | 0.70 | 0.21 | 2 | 0.70 | 0.22 | 2 |
| 85.5 | 0.65 | 0.17 | 2 | 0.67 | 0.17 | 2 | 0.57 | 0.12 | 2 |
| 86.5 | 0.66 | 0.18 | 2 | 0.68 | 0.26 | 2 | 0.59 | 0.37 | 2 |
| 87.5 | 0.66 | 0.16 | 2 | 0.71 | 0.22 | 2 | 0.76 | 0.24 | 2 |
| 88.5 | 0.66 | 0.17 | 2 | 0.66 | 0.24 | 2 | 0.81 | 0.38 | 2 |
| 89.4 | 0.66 | 0.17 | 2 | 0.70 | 0.23 | 2 | 0.76 | 0.26 | 2 |

1. A compound of the formula: 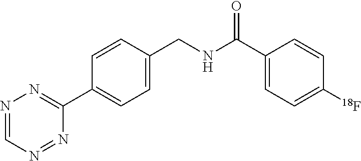 [SMILES: O=C(NCc1ccc(-c2nncnn2)cc1)c1ccc([18F])cc1]
or pharmaceutically acceptable salt thereof.

2. The compound according to claim 1, which is

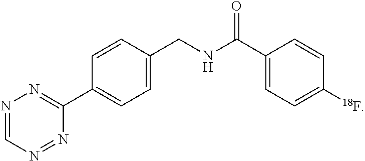

3. A compound of the formula: 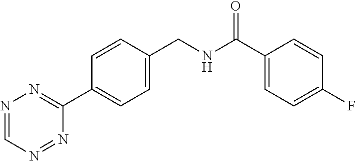 [SMILES: O=C(NCc1ccc(-c2nncnn2)cc1)c1ccc(F)cc1]
or pharmaceutically acceptable salt thereof.

4. The compound according to claim 3, which is

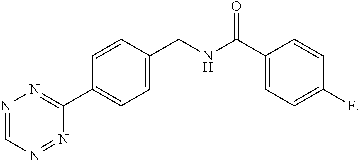 [SMILES: O=C(NCc1ccc(-c2nncnn2)cc1)c1ccc(F)cc1]

5. A pharmaceutical composition comprising a compound or a pharmaceutically acceptable salt thereof according to claim 1, with one or more pharmaceutically acceptable carriers, diluents or excipients.

6. A composition comprising: 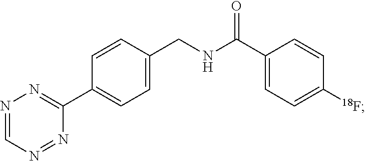
or a pharmaceutically acceptable salt thereof, and ethanol and PBS buffer.

7. The composition of claim 6, wherein the composition does not include ascorbate or ascorbic acid.

8. A method of pretargeted imaging comprising:
a. introducing into a mammal a detectable quantity of the compound: 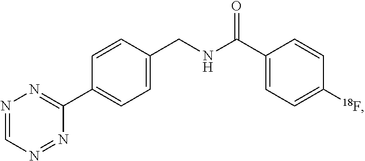
or a pharmaceutically acceptable salt thereof, and
b. detecting said compound in PET imaging, wherein the compound is introduced after a biologics-TCO conjugate was introduced into the mammal.

9. A method of pretargeted imaging comprising:
a. introducing into a mammal a detectable quantity of the composition of claim 5; and
b. detecting the composition.

10. A pharmaceutical composition comprising a compound or a pharmaceutically acceptable salt thereof according to claim 3, with one or more pharmaceutically acceptable carriers, diluents or excipients.

11. A method of pretargeted imaging comprising:
a. introducing into a mammal a detectable quantity of the composition of claim 6; and
b. detecting the composition.
